(19) United States
(12) Patent Application Publication
(10) Pub. No.: US 2007/0196476 A1
(43) Pub. Date: Aug. 23, 2007

(54) Rapidly dissolving tablets comprising low surface area titanium dioxide

(76) Inventors: Michael C. Withiam, Landenberg, PA (US); Dev K. Mehra, Furlong, PA (US); John M. Cornelius, Forest Hill, MD (US)

Correspondence Address: J M HUBER CORPORATION, 333 THORNALL STREET, PATENT DEPARTMENT, EDISON, NJ, 08837-2220, US

(21) Appl. No.: 11/646,152

(22) Filed: Dec. 27, 2006

Related U.S. Application Data
(63) Continuation-in-part of application No. 10/835,666, filed on Apr. 30, 2004, now abandoned.

(51) Int. Cl. A61K 9/46

(52) U.S. Cl. 424/465

(57) ABSTRACT

This invention pertains to the ability to provide rapidly disintegrating tablets through the inclusion of a titanium dioxide material in combination with other common tablet components. Such a titanium dioxide material must exhibit a sufficiently low surface area in order to boost the ability of the tablet to separate quickly when introduced into a user's mouth cavity. Such a tablet is dimensionally stable prior to use (low friability) and, when immersed in water the tablet disintegrates therein in less than about 60 seconds.

### CROSS-REFERENCE TO RELATED APPLICATIONS

This patent application is a continuation-in-part of prior U.S. patent application Ser. No. 10/835,666, filed Apr. 30, 2004, which is hereby incorporated herein by reference in its entirety.

### FIELD OF THE INVENTION

This invention pertains to the ability to provide rapidly disintegrating tablets through the inclusion of a titanium dioxide material in combination with other common tablet components. Such a titanium dioxide material must exhibit a sufficiently low surface area in order to boost the ability of the tablet to separate quickly when introduced into a user's mouth cavity. Such a tablet is dimensionally stable prior to use (low friability) and, when immersed in water the tablet disintegrates therein in less than about 60 seconds.

### BACKGROUND OF THE INVENTION

Many consumer products, such as pharmaceuticals, nutraceuticals, health and personal care products, are manufactured and packaged in solid, compacted form. The solid, compacted product form has several advantages over other product forms, such as relative ease of manufacture and durability in packaging and shipment and convenience in use and in storing for retailers and consumers alike. The compressed tablet form is particularly well-suited for the transfer of medicaments and other treatments to a patient through the oral cavity.

However, in certain situations it would be beneficial if the tablet would disintegrate in the mouth quickly in order to facilitate swallowing by a patient. The young and certain elder patients, as well as people of other ages, may exhibit differing levels of ease in swallowing certain items, particularly tablets. Chewing such products may not be desirable as the taste of medicaments and carriers thereof in such forms are potentially unwanted. Thus, there has been a drive to develop quickly disintegrating tablets for ease in swallowing without chewing but with the reliability of proper delivery of the treatment agent (pharmaceutical, mouth freshener, and the like, without limitation) present therein to the user.

Unfortunately, most tablets do not readily disintegrate in the mouth, but instead disintegrate in a slow and uneven fashion, for example when chewed. Given the forgoing there is a continuing need for solid form oral care preparations that rapidly disintegrate in the mouth and that are not friable under packaging and shipping conditions.

### BRIEF SUMMARY OF THE INVENTION

The present invention includes a rapidly disintegrating tablet comprising (a) about 10% to about 80% of low surface area titanium dioxide material, (b) about 20% to about 80% of a sugar alcohol (c) about 1% to about 30% of a super-disintegrant, and (d) optionally, at least one treatment material selected from the group consisting of a pharmaceutical active, a nutraceutical active, an oral care active, and any combination thereof. Such an inventive tablet provides an effective quick dissolving result while also exhibiting low friability such that the product is highly acceptable to the user aesthetically as well. Without the low surface area calcium phosphate material, the resultant tablet would not exhibit the same degree of quick dissolution.

### DETAILED DESCRIPTION OF THE INVENTION

All parts, percentages and ratios used herein are expressed by weight unless otherwise specified.

All publications, patent applications and issued patents mentioned herein are hereby incorporated in their entirety by reference.

The present invention relates to any number of treatment agents that are delivered via tablet forms. Thus, pharmaceuticals (medicines, for instance), nutraceuticals (vitamins, mineral supplements, and the like), breath fresheners, tooth cleaners, and the like.

The tablets of this invention would include, in addition to the treatment agents noted above, from about 10% to about 80% of the low surface area titanium dioxide (3.0 to 10.0 m²/g, preferably from 4 to 6 m²/g, most preferably about 5), preferably from about 15% to about 50%, about 20% to 80% sugar alcohol, preferably about 20% to about 70%, and about 1% to about 30% of a super disintegrant, preferably about 3% to about 15%, more preferably about 3% to 5%.

The low surface area titanium dioxide component of the inventive tablet substrate exhibits a surface area from 3.0 to 10.0 m²/g, preferably from 4 to 6 m²/g, most preferably about 5. This low surface area is required to provide the quick disintegration properties of the inventive tablets in combination with the other required components therein. Suitable titanium dioxide may be rutile or anatase forms, which are often derived from ilmenite or leuxocene ores. A suitable source of titanium dioxide is a 5.0 m²/g Tronox® titanium dioxide available from Kerr-McGee Pigments Oklahoma City, Okla.

The sugar alcohol provides multiple functions to the rapidly disintegrating tablet. The sugar alcohol provides good aesthetic properties to the dissolved oral care tablet such as taste and “mouth texture” or body; aids in rapid tablet disintegration; and serves as a tablet filler. Suitable sugar alcohols include glycerin (glycerol), erythritol, xylitol, sorbitol, maltitol, mannitol, lactitol, and the like, used singly and in combinations, with mannitol and sorbitol preferred.

The super disintegrant facilitates the break-up of a tablet when it is placed in an aqueous environment, such as the mouth. Super disintegrants in contact with water swell, wick-in water or otherwise provide a disruptive force to a tablet causing it to break apart. Suitable super disintegrants include one or more of sodium starch glycolate, available as e.g. Explotab and Explosol; croscarmellose sodium (cross-linked sodium carboxymethyl cellulose) available as e.g. Ac-Di-Sol® and Nymcel® ZSX; and cross-linked polyvinylpyrolidones available as e.g. Polyplasdone XL.

In addition to the aforementioned ingredients, the tablet products of the present invention may also include several other ingredients such as additional disintegration aids, organoleptic enhancers, additional abrasives, thickening agents, (also sometimes known as thickeners, binders, gums, or stabilizing agents), therapeutic agents, and preservatives.

These solid formed tablet preparations may also include one or more disintegration aids, in addition to the super disintegrant. Suitable disintegration aids include natural, modified or pregelatinized starch; natural or chemically-modified cellulose; microcrystalline cellulose; gum, especially agar gum, and guar gum; alginic acid or salts thereof; acetates and citrates; sugars (especially sucrose, amylose, dextrose and lactose); aluminum oxide; synthetic polymers such as methacrylic acid-divinylbenzene copolymer, as well as effervescent disintegrating systems. Typical levels of disintegration aids in the inventive tablet preparations are from about 0.5% to about 15% of the formulation, preferably from about 1% to about 5%.

The inventive tablet compositions may also contain one or more organoleptic enhancing agents. Organoleptic enhancing agents include humectants, sweeteners, surfactants, flavorants, colorants and effervescing agents.

Humectants serve to add body or “mouth texture” to a tablet. In addition to the previously mentioned sugar alcohols, suitable humectants include glycerin, polyethylene glycol (at a variety of different molecular weights), propylene glycol, and hydrogenated starch hydrolyzates, as well as mixtures of these compounds.

Sweeteners may be added to the tablet composition to impart a pleasing taste to the product. Suitable sweeteners include saccharin (as sodium, potassium or calcium saccharin), cyclamate (as a sodium, potassium or calcium salt), aspartame, acesulfane-K, thaumatin, neohisperidin dihydrochalcone, ammoniated glycyrrhizin, dextrose, maltodextrin, sucralose, fructose, levulose, sucrose, mannose, and glucose. Typical levels of sweeteners are from about 0% to about 5% of a tablet composition.

Surfactants are used in the compositions of the present invention to make the compositions more cosmetically acceptable. The surfactant is preferably a detersive material which imparts to the composition detersive and foaming properties. Suitable surfactants are safe and effective amounts of anionic, cationic, nonionic, zwitterionic, amphoteric and betaine surfactants such as sodium lauryl sulfate, sodium dodecyl benzene sulfonate, alkali metal or ammonium salts of lauroyl sarcosinate, myristoyl sarcosinate, palmitoyl sarcosinate, stearoyl sarcosinate and oleoyl sarcosinate, polyoxyethylene sorbitan monostearate, isostearate and laurate, sodium lauryl sulfoacetate, N-lauroyl sarcosine, the sodium, potassium, and ethanolamine salts of N-lauroyl, N-myristoyl, or N-palmitoyl sarcosine, polyethylene oxide condensates of alkyl phenols, cocoamidopropyl betaine, lauramidopropyl betaine, palmityl betaine and the like. Sodium lauryl sulfate is a preferred surfactant. The surfactant is typically present in the tablet compositions of the present invention in an amount of about 0.1 to about 15% by weight, preferably about 0.3% to about 5% by weight, such as from about 0.3% to about 2%, by weight.

Flavoring agents optionally can be added to tablet compositions. Suitable flavoring agents include, but are not limited to, oil of wintergreen, oil of peppermint, oil of spearmint, oil of sassafras, and oil of clove, cinnamon, anethole, menthol, thymol, eugenol, eucalyptol, lemon, orange and other such flavor compounds to add fruit notes, spice notes, etc. These flavoring agents consist chemically of mixtures of aldehydes, ketones, esters, phenols, acids, and aliphatic, aromatic and other alcohols.

Colorants may be added to improve the aesthetic appearance of the tablet product. Suitable colorants are selected from colorants approved by appropriate regulatory bodies such as the FDA and those listed in the European Food and Pharmaceutical Directives and include pigments, such as TiO₂, and colors such as FD&C and D&C dyes.

The tablet product may also contain an effervescent agent to provide aesthetic properties to the tablet. Preferably effervescence is provided by reaction of a carbonate salt such as calcium carbonate, sodium carbonate, sodium bicarbonate, potassium carbonate or potassium bicarbonate with an acid such as citric acid, tartaric acid or malic acid.

Thickening agents are useful in the tablet products of the present invention to provide an aesthetically pleasing texture when the composition disintegrates in the mouth. Suitable thickening agents include silica thickeners such as J.M. Huber Corporation Zeodent® precipitated silica products and silica gels available from Davison Chemical Division of W. R. Grace Corporation, Baltimore, Md.; natural and synthetic clays such as hectorite clays; lithium magnesium silicate (laponite) and magnesium aluminum silicate (Veegum); starch; glycerite of starch; as well as mixtures of these compounds. Typical levels of thickening agents are from about 0% to about 15% of an oral care composition.

The tablet will contain at least one treatment agent selected from pharmaceutical actives, nutraceutical actives, and oral care actives. Pharmaceutical actives will impart medicinal treatments to a user through ingestion in the mouth. The active substances which can be used according to the invention may be selected without limitation among those belonging to the following groups:

analgesic drugs such as, e.g., buprenorphine, codeine, fentanyl, morphine, hydromorphone, and the like; anti-inflammatory drugs such as, e.g., ibuprofen, indomethacin, naproxen, diclofenac, tolfenamic acid, piroxicam, and the like; anthelmintics such as albendazole, flubendazole, ivermectin, diethylcarbamazine citrate and the like. Antibacterials such as aminoglycosides (Kanamycin, Neomycin, and the like), Rifampin, cephalosporins and related beta lactams (Cefazolin, Cefuroxime, Cefaclor and the like), glycopeptides (Vancomycin and the like), penicillins (amoxicillin, ampicillin, carbenecillin, cloxacillin, dicloxacillin, and the like), quinolones (gatifloxcin, ciprofloxacin and the like), sulfonamides (sulfadiazine, sulfamethoxazole, sulfamerazine, trimethoprim, sulfanilamide, and the like), tranquilizers such as, e.g., diazepam, droperiodol, fluspirilene, haloperidol, lorazepam, and the like; cardiac glycosides such as, e.g., digoxin, ouabain, and the like; antiparkinson agents such as, e.g., bromocriptine, biperidin, benzhexol, benztropine, and the like; antidepressants such as, e.g., imipramine, nortriptyline, pritiptylene, lithium carbonate, clozapine, citalopram, fluoxeitine and the like; antineoplastic agents and immunosuppressants such as, e.g., cyclosporin A, fluorouracil, mercaptopurine, methotrexate, mitomycin, and the like; antiviral agents such as, e.g., idoxuridine, acyclovir, vidarabin, and the like; antibiotic agents such as, e.g., clindamycin, erythromycin, fusidic acid, gentamicin, and the like; antifungal agents such as, e.g., miconazole, ketoconazole, clotrimazole, amphotericin B, nystatin, and the like; antimicrobial agents such as, e.g., metronidazole, tetracyclines, and the like; appetite suppressants such as, e.g., fenfluramine, mazindol, phentermin, and the like; antiemetics such as, e.g., metoclopramide, droperidol, haloperidol, promethazine, and the like; antihistamines such as, e.g., chlorpheniramine, chlorpheniramine maleate,terfenadine, triprolidine, and the like; antimigraine agents such as, e.g., dihydroergotamine, ergotamine, pizotyline, and the like; coronary, cerebral or peripheral vasodilators such as, e.g., nifedipine, diltiazem, and the like; antianginals such as, e.g., glyceryl nitrate, isosorbide dinitrate, molsidomine, verapamil, and the like; calcium channel blockers such as, e.g., verapamil, nifedipine, diltiazem, nicardipine, and the like; hormonal agents such as, e.g., estradiol, estron, estriol, polyestradiol, polyestriol, dienestrol, diethylstilbestrol, progesterone, dihyroergosterone, cyproterone, danazol, testosterone, and the like; contraceptive agents such as, e.g., ethinyl estradiol, lynestrenol, etynodiol, norethisterone, mestranol, norgestrel, levonorgestrel, desogestrel, edroxyprogesterone, and the like; antithrombotic agents such as, e.g., warfarin, and the like; diuretics such as, e.g., hydrochlorothiazide, flunarizine, minoxidil, and the like; antihypertensive agents such as, e.g., propanolol, metoprolol such as metoprolol tartrate or metoprolol succinate, clonidine, pindolol, and the like; chemical dependency drugs such as, e.g., nicotine, methadone, and the like; local anesthetics such as, e.g., prilocaine, benzocaine, and the like; corticosteroids such as, e.g., beclomethasone, betamethasone, clobetasol, desonide, desoxymethasone, dexamethasone, diflucortolone, flumethasone, fluocinolone acetonide, fluocinonide, hydrocortisone, ethylprednisolone, triamcinolone acetonide, budesonide, halcinonide, and the like; dermatological agents such as, e.g., nitrofurantoin, dithranol, clioquinol, hydroxyquinoline, isotretionin, methoxsalen, methotrexate, tretionin, trioxsalen, salicylic acid, penicillamine, and the like; steroids such as, e.g., estradiol, progesterone, norethindrone, levonorgestrol, ethynodiol, levenorgestrel, norgestimate, gestanin, desogestrel, 3-keton-desogestrel, demegestone, promethoestrol, testosterone, spironolactone, and esters thereof, azole derivatives such as, e.g., imidazoles and mazoles and derivatives thereof, nitro compounds such as, e.g., amyl nitrates, nitroglycerine and isosorbide nitrates, amine compounds such as, e.g., pilocaine, oxyabutyninchloride, benzocaine, nicotine, chlorpheniramine, terfenadine, triprolidine, propanolol, metoprolol and salts thereof, oxicam derivatives such as, e.g., piroxicam, mucopolysaccharides such as, e.g., thiomucasee, opoid compounds such as, e.g., morphine and morphine-like drugs such as buprenorphine, oxymorphone, hydromorphone, levorphanol, hydrocodone, hydrocodone bitratrate, fentanyl and fentany derivatives and analogues, prostaglandins such as, e.g., a member of the PGA, PGB, PGE, or PGF series such as, e.g., misoprostol or enaprostil, a benzamide such as, e.g., metoclopramide, scopolamine, a peptide such as calcitonin, serratiopeptidase, superoxide dismutase (SOD), tryrotropin releasing hormone (TRH), growth hormone releasing hormone (GHRH), and the like, a xanthine such as, e.g., caffeine, theophylline, a catecholamine such as, e.g., ephedrine, salbutamol, terbutaline, a dihydropyridine such as, e.g., nifedipine, a thiazide such as, e.g., hydrochlorotiazide, flunarizine, a sydnonimine such as, e.g., molsidomine, and a sulfated polysaccharide, as well as cholesterol-lowering statin drugs, such as atorvastatin, simvastatin, and the like.

The active substances mentioned above are also listed for illustrative purposes; the invention is applicable to any pharmaceutical formulation regardless of the active substance or substances incorporated therein. They can be present in any amount, but preferably from 0.01 to about 30% by weight therein.

Typical nutraceutical actives include vitamins (any of the typical ones, such as Vitamins A, B₆, B₁₂, C, D, and K) as well as mineral supplements (calcium carbonate, calcium phosphate, and other types of compounds that deliver desirable doses of calcium, magnesium, and other like minerals to a user). The same proportion of nutraceutical active as for the pharmaceutical types may be present.

Typical oral care actives include abrasives. Suitable abrasives include precipitated and ground calcium carbonate, calcium metasilicate, calcium pyrophosphate, dicalcium phosphate, dicalcium phosphate dihydrate, aluminum silicate, alumina, calcined alumina, bentonite, particulate thermosetting resins and other suitable abrasive materials known to a person of ordinary skill in the art. The abrasive may be used alone or in combination with other abrasives. Typical levels of abrasives in the inventive dentifrice formulation are from about 2% to about 60%, preferably from about 2% to about 10%.

Further oral care actives include various therapeutic agents for the prevention and treatment of dental caries, periodontal disease and temperature sensitivity. Examples of therapeutic agents, without intending to be limiting, are fluoride sources, such as sodium fluoride, sodium monofluorophosphate, stannous fluoride, potassium fluoride, sodium fluorosilicate, ammonium fluorosilicate and the like; condensed phosphates such as tripolyphosphates, hexametaphosphates, trimetaphosphates and pyrophosphates; antimicrobial agents such as triclosan, bisguanides, such as alexidine, chlorhexidine and chlorhexidine gluconate; enzymes such as papain, bromelain, glucoamylase, amylase, dextranase, mutanase, lipases, pectinase, tannase, and proteases; quarternary ammonium compounds, such as benzalkonium chloride (BZK), benzethonium chloride (BZT), cetylpyridinium chloride (CPC), and domiphen bromide; metal salts, such as zinc citrate, zinc chloride, and stannous fluoride; sanguinaria extract and sanguinarine; volatile oils, such as eucalyptol, menthol, thymol, and methyl salicylate; amine fluorides; peroxides and the like. Therapeutic agents may be used in dentifrice formulations singly or in combination at a therapeutically safe and effective level.

Preservatives may be also be optionally added to the compositions of the present invention to prevent bacterial growth. Suitable preservatives approved for use in oral compositions such as methylparaben, propylparaben and sodium benzoate may be added in safe and effective amounts.

The tablet products may additionally contain other optional ingredients typically used in tablet making such as glidants to provide even flow to the granulation to be tabletted, e.g. amorphous silica such as Zeopharm® 80 (J.M. Huber Corporation, Edison, N.J.) and Cab-O-Sil® M5 (Cabot Corporation, Billerica, Mass.); die release aids, also known as lubricants, such as magnesium stearate (available as HYQUAL® NF from Mallinckrodt, Inc., St. Louis, Mo.) to enable tablets to be released from within the tablet machine die, anti-adherents, such as stearic acid, to facilitate separation of tablets from punch faces; and fillers such as microcrystalline cellulose, such as Avicel 101 (FMC Biopolymers, Philadelphia, Pa.) and Omnicel 102 (Functional Foods, Englishtown, N.J.).

All tablet formulation ingredients, except the lubricant, are weighed together and mixed. Thereafter, the lubricant is geometrically diluted with the just prepared tablet mixture and then added back to the mixture. This step is typically necessary to homogeneously incorporate the hydrophobic lubricant into the tablet mixture.

The tablets are then manufactured by using a tableting compacting process. A standard single stroke or a rotary press may be used. The tablets prepared according to this invention may be of any geometrical shape, such as round, square, triangular, or caplet-shaped, and of any size suitable for human or animal use.

The invention will now be described in more detail with respect to the following, specific, non-limiting examples.

### Preferred Embodiments of the Invention

### Tablet Preparation

Tablets were prepared by weighing all formulation ingredients together, except the lubricant magnesium stearate, on a weighing pan. Typically, a tablet formulation was 300 g to 500 g total weight, in order to prepare multiple tablets for testing. The combined ingredients were passed through a 20 mesh (850 μm) sieve to remove any lumps and then bag blended, by gentle inversion in a plastic bag for about 30 seconds of the formulation ingredients previously weighed. The resulting mixture was transferred to a PK-V blender (twin shell dry blender model 014-215-0053, available from Patterson Kelly, East Stroudsburg, Pa.) and mixed for 10 minutes. The magnesium stearate lubricant was then geometrically diluted with the mixture and then added back to the PK blender and all ingredients mixed together for an additional 5 minutes.

Tablets were formed from the resulting formulation on a 8-station Piccola rotary tablet press available from Riva S.A., Argentina, fitted with 10 mm standard concave die punches compacting over a range of compression forces. Tablet weight was set at 400 mg by adjusting the tablet press.

### Tablet Test Methods

All tablets were prepared 24 hours before testing hardness, disintegration time and friability.

Tablet hardness (H) expressed in kP, for each formulation, was measured on 5 tablets utilizing a Erweka TBH30 instrument (Milford, Conn.) and the result reported was an average of 5 measurements.

Tablet disintegration time was determined, according to the USP test for uncoated tablets by placing 6 tablets (each in a separate tube) in an Erweka ZT72 disintegrator (Milford, Conn.). The tablets were repeatedly immersed in 37° C. deionized water at a rate of 30 strokes/min. until the tablets disintegrated, as detected and recorded by the instrument. The reported result was an average of the 6 measurements.

Tablet friability was determined by placing 10 tablets in a Distek, Inc. Friabilator DF-3 (North Brunswick, N.J.) set for 100 revolutions. The % friability is calculated from the amount of tablet weight lost (friable) by weighing the tablets before and after rotation.

### EXAMPLES 1-4

In theses examples, tablet formulations were made with titanium dioxide (TiO₂), a super disintegrant, a sugar alcohol and other ingredients typically found in oral care formulations and in pharmaceutical tablet formulations. Formulations 1-3 represent placebo pharmaceutical tablet formulations. An active pharmaceutical ingredient could be substituted for a portion of the microcrystalline cellulose, mannitol and titanium dioxide, depending on the dosage desired. Formulations 3 and 4 are typical oral care tablet formulations containing ingredients typically found in oral care formulations, such as a surfactant, additional abrasive, an enzyme and sodium fluoride. These formulations were prepared according to the procedure described above with the amounts of ingredients identified in Table 1.

TABLE 1
Tablet Formulations

| | | Formulation No. | | | |
|---|---|---|---|---|---|
| | Source | 1 | 2 | 3 | 4 |
| TiO₂, % | Kerr-McGee | 40 | 25 | 10 | 20 |
| Tronox | Pigments | | | | |
| | Oklahoma City, OK | | | | |
| Mannitol, % | Roquette Freres | 38.25 | 53.25 | 63.25 | 0 |
| Pearlitol 200SD | Lestrem, France | | | | |
| Sorbitol, % | Sigma Chemicals | 0 | 0 | 0 | 8 |
| D-Sorbitol, min 98% | | | | | |
| MCC, % | Functional Foods | 15 | 15 | 20 | 16.39 |
| Omnicel 102 | Englishtown, NJ | | | | |
| Crospovidone, % | ISP Technologies, | 0 | 0 | 5 | 14 |
| Polyplasdone XL | Inc. Wayne, NJ | | | | |
| ExploTab, % | Penwest | 2.5 | 5 | 0 | 0 |
| Sodium starch | Pharmaceuticals | | | | |
| glycolate | Patterson., NY | | | | |
| Nymcel ZSK | Noviant | 2.5 | 0 | 0 | 0 |
| Croscarmellose | The Netherlands | | | | |
| sodium | | | | | |
| Sodium Lauryl | | 0 | 0 | 1 | 1 |
| Sulfate, % | | | | | |
| Cab-O-Sil M-5, % | Cabot Corporation | 1 | 1 | 1 | 1 |
| Silica glidant | Billerica, MA | | | | |
| Sodium fluoride, % | Sigma Chemicals | 0 | 0 | 0 | 0.01 |
| Papain, % | National Enzyme | 0 | 0 | 0 | 0.1 |
| | Forsyth, MO | | | | |
| Aspartame, % | Ajinomoto Co. | 0 | 0 | 0 | 3 |
| | Japan | | | | |
| Flavor, % | Invetech | 0 | 0 | 0 | 3 |
| Citrus blend | Tustin, CA | | | | |
| Sodium | Arm & Hammer | 0 | 0 | 0 | 20 |
| bicarbonate, % | | | | | |
| Citric Acid, % | | 0 | 0 | 0 | 10 |
| Zeodent 9175, % | J. M. Huber Corp. | 0 | 0 | 0 | 3 |
| silica abrasive | Edison, NJ | | | | |
| Magnesium | Malinckrodt | 0.75 | 0.75 | 0.75 | 0.5 |
| Stearate, % | St. Louis, MO | | | | |

Tablets weighing 400 mg each were prepared according to the procedure described above. Each formulation was compressed into tablets at different compression forces for each respective formulation. The tablet hardness (H), disintegration time (DT) and Friability were determined according to the procedures described above for tablets pressed at different compression forces with the results summarized in Table 2 below.

TABLE 2
Tablet Properties

| Formulation | | | |
|---|---|---|---|
| No. | H (kP) | DT (sec) | % Friability |
| 1 | 2.75 | 24 | 1.45 |
| 1 | 4.14 | 16 | 1.45 |
| 2 | 3.28 | 28 | 0.60 |
| 2 | 7.03 | 23 | 0.35 |
| 2 | 7.34 | 33 | 0.2 |
| 3 | 3.18 | 8 | 0.785 |
| 3 | 7.73 | 12 | 0.278 |
| 3 | 11.31 | 15 | 0.176 |
| 4 | 2.82 | 41 | 0.71 |

It seen from the data above that all formulations provided tablets that could be compressed to an acceptable hardness providing friability of less than 2% and rapid disintegration times of no greater than about 40 seconds. This small friability percentage reflects the fact that the tablets, despite their very fast disintegration times, are also strong and have excellent physical integrity. This means that they can remain intact during the periods of storage and transportation until being finally delivered to the consumer.

### COMPARATIVE EXAMPLE

For comparison, a tablet formulation containing the sugar alcohol mannitol and magnesium stearate lubricant, but no titanium dioxide and no super disintegrant, labeled Formulation C was prepared as described above. The formulation is given in Table 3 below.

TABLE 3
Comparative Example Tablet Formulation

| | Source | Formulation C |
|---|---|---|
| Mannitol, % | Roquette Freres | 99.25 |
| Pearlitol 200SD | Lestrem, France | |
| Magnesium | Malinckrodt | 0.75 |
| Stearate, % | | |

Tablets of Formulation C were made according to the procedure described above by compressing the tablets with different forces to provide tablets of differing hardness. These tablets were tested for hardness and disintegration time (DT) according to the methods previously described.

TABLE 4
Performance

| | Hardness (kP) | DT (s) |
|---|---|---|
| Formulation C | 3.5 | 42 |
| Formulation C | 10.0 | 165 |
| Formulation C | 13.4 | 145 |

It is seen from the above data that tablets without titanium dioxide and without a super disintegrant had longer disintegration times than tablets of comparable hardness made according to the present invention.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

What we claim is:

1. An orally-administered rapidly disintegrating tablet comprising:
a titanium dioxide exhibiting a surface area of from 3.0 to 10.0 m²/g;
a super disintegrant;
a sugar alcohol; and, optionally
at least one treatment agent selected from the group consisting of a pharmaceutical active, a nutraceutical active, an oral care active, and any combinations thereof;
wherein said tablet exhibits a friability of less than about 2% and disintegrates when immersed in water in less than about 60 seconds.

2. The orally-administered tablet of claim 1 wherein said calcium carbonate exhibits a surface area of from 4.0 to 6.0 m²/g.

3. The orally-administered tablet of claim 1 wherein said calcium carbonate exhibits a surface area of about 5.0 m²/g.

6. The orally-administered tablet of claim 1, wherein the tablet comprises about 10% to about 80 wt % of titanium dioxide.

7. The orally-administered tablet of claim 1, wherein the super disintegrant is selected from one or more of sodium starch glycolate, croscarmellose sodium, and crospovidone.

8. The orally-administered tablet of claim 1, wherein the tablet comprises about 1 wt % to about 30 wt % of the super disintegrant.

9. The orally-administered tablet of claim 1, wherein the tablet comprises about 1 wt % to about 3 wt % of the super disintegrant.

10. The orally-administered tablet of claim 1, wherein the sugar alcohol is selected from one or more of sorbitol, mannitol, xylitol, erythritol, maltitol, and lactitol.

9. The orally-administered tablet of claim 1, wherein the tablet comprises about 20 wt % to about 80 wt % of the sugar alcohol.

10. The orally-administered tablet of claim 1, wherein the tablet friability is less than 1%.

11. The orally-administered tablet of claim 1, wherein the tablet, when added to water at 37° C. disintegrates in less 40 seconds.

12. The orally-administered tablet of claim 1, further comprising one or more ingredients selected from the group consisting of organoleptic enhancing agents, disintegration aids, preservatives, surfactants and thickening agents.

13. The orally-administered tablet of claim 13 wherein the organoleptic enhancing agent is selected from the group consisting of humectants, sweeteners, flavorants, surfactants, colorants and effervescent agents.

14. The orally-administered tablet of claim 1, wherein the tablet further comprises said pharmaceutical active.

15. An orally-administered, rapidly disintegrating tablet comprising:
about 10 wt % to about 80 wt % titanium dioxide exhibiting a surface area of from 3.0 to 10.0 m²/g;
about 1 wt % to about 15 wt % super disintegrant;
about 20 wt % to about 80 wt % sugar alcohol;
about 0.1 wt % to about 5 wt % surfactant; and, optionally
at least one treatment agent selected from the group consisting of a pharmaceutical active, a nutraceutical active, an oral care active, and any combinations thereof;
wherein said tablet exhibits a friability of less than about 2% and the tablet disintegrates when immersed in water in less than about 60 seconds.

16. The orally-administered tablet of claim 15 further comprising a flavorant.
